(19) United States
(12) Patent Application Publication
(10) Pub. No.: US 2005/0277784 A1
(43) Pub. Date: Dec. 15, 2005

(54) Processes for the preparation of 2-cyano-3-naphthalene-1-yl-acrylic acid alkyl or benzyl esters

(75) Inventors: Weiguo Liu, Parsippany, NJ (US); John L. Considine, Bridgewater, NJ (US); Zhixian Ding, Fort Lee, NJ (US); John Potoski, West Nyack, NY (US)

Correspondence Address: COZEN O' CONNOR, P. C., 1900 MARKET STREET, PHILADELPHIA, PA, 19103-3508, US

(73) Assignee: Wyeth, Madison, NJ (US)

(21) Appl. No.: 11/148,603

(22) Filed: Jun. 9, 2005

Related U.S. Application Data
(60) Provisional application No. 60/579,295, filed on Jun. 14, 2004.

(51) Int. Cl. C07C 253/30

(52) U.S. Cl. 558/357

(57) ABSTRACT

The present invention provides processes for the preparation of compounds of Formula III that are useful in the preparation of pharmaceuticals for the treatment of inflammatory diseases.

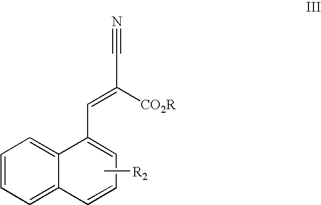

### CROSS REFERENCE TO RELATED APPLICATIONS

This application claims priority benefit of U.S. Provisional Application Serial No. 60/579,295, filed Jun. 14, 2004, which is hereby incorporated by reference in its entirety.

### FIELD OF THE INVENTION

The present invention relates to processes for the preparation of 2-cyano-3-naphthalene-1-yl-acrylic acid alkyl esters useful as chemical intermediates for the preparation of 3-(1-naphthyl)-3-phenyl-2-cyanopropanoic acid amides and esters useful in the treatment of inflammation and other diseases.

### BACKGROUND OF THE INVENTION

Ligands of the estrogen receptor (ER) have been shown to inhibit inflammatory gene expression that typically causes a reduction of cytokines, chemokines, adhesion molecules and other inflammatory enzymes. Accordingly, ER ligands can provide a means to treat inflammation such as the inflammatory component of diseases including, for example, atherosclerosis, myocardial infarction (MI), congestive heart failure (CHF), inflammatory bowel disease and arthritis. Other potential therapeutic indications for these type of molecules include type II diabetes (Cefalu, J Womens Health & Gender-based Med., 2001, 10, 241; Yuan et al., Science, 2001, 293, 1673), osteoarthritis (Pelletier et al., Arthr. & Rheum., 2001, 44:1237; Felson et al., Curr Opinion Rheum, 1998, 10, 269) asthma (Chin-Chi Lin et. al., Immunol. Lett., 2000, 73, 57), Alzheimer's disease (Roth, A. et. al.,; J. Neurosci. Res., 1999, 57, 399) and autoimmune diseases such as multiple sclerosis and rheumatoid arthritis.

A common component of chronic inflammatory conditions is polymorphonuclear leukocyte and monocyte infiltration into the site of damage through increased expression of cytokines and adhesion molecules responsible for their recruitment. Overproduction of the cytokine interleukin (IL-6) has been associated with states of chronic inflammation (Bauer M. A., Herrmann F., Ann. Hematol., 1991, 62, 203). Synthesis of the IL-6 gene is induced by the transcription factor, nuclear factor κB (NF-κB). Interference at this step in the inflammatory process can effectively regulate the uncontrolled proliferative process that occurs in these chronic conditions.

In endothelial cells, 17β-estradiol (E2) inhibits IL-1β induced NF-κB reporter activity and IL-6 expression in an ER dependent fashion (Kurebayashi S. et. al., J. Steroid Biochem. Molec. Biol., 1997, 60, 11). This activity correlates with anti-inflammatory action of E2 in vivo as confirmed in different animal models of inflammation. In models of atherosclerosis, E2 was shown to protect endothelial cell integrity and function and to reduce leukocyte adhesion and intimal accumulation (Adams, M. R. et al., Arterio., 1990, 1051; Sullivan, T. R. et al. J. Clin. Invst., 1995, 96, 2482; Nathan, L. et. al., Circ. Res., 1999, 85, 377). Similar effects of estrogen on the vascular wall also have been demonstrated in animal models of myocardial infarction (Delyani, J. A. et al., J. Molec. Cell. Cardiol., 1996, 28, 1001) and congestive heart failure. Clinically, estrogen replacement therapy (ERT) has been demonstrated to reduce the risk of mortality in patients with both CHF (Reis et. al., J. Am. Coll. Cardio., 2000, 36, 529) and Ml (Grodstein, F. et. al., Ann. Int. Med., 2000, 133, 933; Alexander et. al., J. Am. Coll. Cardio., 2001, 38, 1; Grodstein F. et. al., Ann. Int. Med, 2001, 135, 1). In ERT, clinical studies demonstrated an influence of E2 on the decrease in the production of β-amyloid 1-42 (Aβ42), a peptide central for the formation of senile plaques in Alzheimer's disease (Schonknecht, P. et. al., Neurosci. Lett., 2001, 307, 122).

3-(1-Naphthyl)-3-phenyl-2-cyanopropanoic acid amides have been shown to activate certain ER pathways and have anti-inflammatory activity as described in, for example, U.S. patent application Ser. No. 10/883,678, filed Apr. 28, 2004, incorporated herein by reference in its entirety. These compounds are useful in treating numerous diseases and disorders characterized as having, for example, an inflammatory component.

Preparation of the above anti-inflammatory amides can be achieved by employing, for example, 2-cyano-3-naphthalene-1-yl-acrylic acid alkyl or benzyl esters as intermediates. Synthetic routes to these acrylic acid alkyl or benzyl ester intermediates have been reported by Fadda, et al. (Indian Journal of Chemistry, 1990, 29B, 171) and Gearien et al. (J. Am. Pharm. Assoc., 1959, 48, 61). However, these routes involve undesirable distillation steps and/or the use of flammable, non-polar extraction solvents. Accordingly, new and improved methods for the preparation of 2-cyano-3-naphthalene-1-yl-acrylic acid alkyl or benzyl esters are needed. The processes and intermediates provided herein can help meet these and other needs.

### SUMMARY OF THE INVENTION

The present invention provides a process for preparing a compound of Formula III: 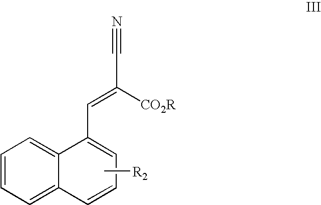
wherein:

R is C₁₋₆ alkyl or benzyl;

R₂ is hydrogen, halogen, C₁₋₆alkyl, C₁₋₆ alkoxy, nitro, cyano, aryl, CF₃, OCF₃, NR4R₅ or OH;

R₄ and R₅ are each, independently, hydrogen, C₁₋₆ alkyl, aryl, arylalkyl having 1-6 carbon atoms in the alkyl moiety, Het-alkyl having 1-6 carbon atoms in the alkyl moiety, hydroxyalkyl of 1-6 carbons, dihydroxyalkyl of 1-6 carbons, or cycloalkyl of 3-7 carbons,

or R₄ and R₅ together with the N atom to which they are attached form a 5- or 6-membered heterocycle; and

Het is a heterocyclic ring system of 4-14 ring atoms comprising one to four ring-forming heteroatoms;
comprising reacting a compound of Formula I: 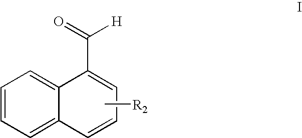
in a protic solvent with a compound of Formula II:

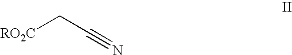

In some embodiments, R is C₁₋₆ alkyl and R₂ is hydrogen or C₁₋₄ alkyl.

In some embodiments, R is C₁₋₄ alkyl and R₂ is hydrogen.

In some embodiments, R is methyl and R₂ is hydrogen.

In some embodiments, the reaction is carried out in the presence of a catalytic amount of secondary amine. Some example secondary amines include pyrrolidine, piperidine, and homopiperidine. In some embodiments, the secondary amine is present in the reaction mixture in an amount that is less than 10 mol % relative to the amount of the compound of Formula I. In further embodiments, the secondary amine is present in the reaction mixture in an amount that is less than 3 mol % relative to the amount of said compound of Formula I.

In some embodiments, the protic solvent comprises an organic alcohol such as methanol, ethanol, iso-propyl alcohol, or any combination thereof. In some embodiments, the organic alcohol is methanol.

In some embodiments, the processes of the invention can further comprise the step of isolating the compound of Formula III by filtration of the reaction mixture.

### DETAILED DESCRIPTION OF EMBODIMENTS OF THE INVENTION

The present invention is directed to, inter alia, processes for the preparation of compounds of Formula III that are useful as intermediates in the preparation of 3-(1-naphthyl)-3-phenyl-2-cyanopropanoic acid amides (see, e.g., U.S. patent application Ser. No. 10/883,678, filed Apr. 28, 2004, incorporated herein by reference in its entirety), which, in turn, are useful in the treatment of inflammatory disorders and other diseases. According to some embodiments, the present invention provides methods of preparing 3-(1-naphthyl)-3-phenyl-2-cyanopropanoic acrylic acid alkyl and benzyl esters without the use of certain non-polar solvents such as benzene or toluene. The processes of the invention also can be conducted without the use of any metal catalyst or the azeotropic removal of water from the reaction medium. In further embodiments, compounds of Formula III can be isolated from the reaction mixture without recourse to extraction techniques using flammable and/or expensive organic solvents.

A general outline of the processes of the present invention is provided in Scheme I, where constituent members of the depicted compounds of Formulas I, II, and III are defined hereinbelow.

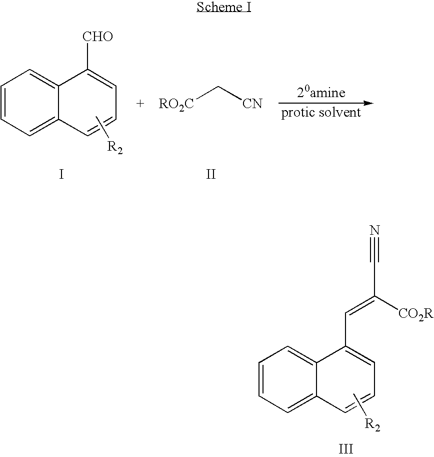

The present invention provides a process for preparing a compound of Formula III: 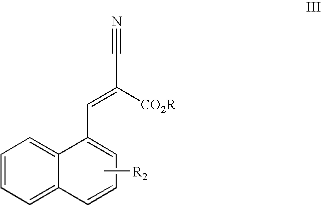
wherein:

R is C₁₋₆ alkyl or benzyl;

R₂ is hydrogen, halogen, C₁₋₆ alkyl, C₁₋₆ alkoxy, nitro, cyano, aryl, CF₃, OCF₃, NR₄R₅ or OH;

R₄ and R₅ are each, independently, hydrogen, C₁₋₆alkyl, aryl, arylalkyl having 1-6 carbon atoms in the alkyl moiety, Het-alkyl having 1-6 carbon atoms in the alkyl moiety, hydroxyalkyl of 1-6 carbons, dihydroxyalkyl of 1-6 carbons, or cycloalkyl of 3-7 carbons,

or R₄ and R₅ together with the N atom to which they are attached form a 5- or 6-membered heterocycle; and

Het is a heterocyclic ring system of 4-14 ring atoms comprising one to four ring-forming heteroatoms;

comprising reacting a compound of Formula I: 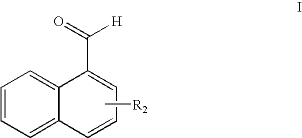
in a protic solvent with a compound of Formula II:

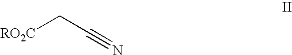

According to some embodiments, R is C₁₋₆ alkyl and R₂ is hydrogen or C₁₋₄ alkyl.

According to some embodiments, R is C₁₋₄ alkyl and R₂ is hydrogen.

According to some embodiments, R is methyl and R₂ is hydrogen.

In further embodiments, the reacting is carried out in the presence of a catalytic amount of secondary amine. Some example secondary amines include pyrrolidine, piperidine, and homopiperidine. In some embodiments, the secondary amine is present in the reaction mixture in an amount that is less than 10 mol % relative to the amount of the compound of Formula I. In further embodiments, the secondary amine is present in the reaction mixture in an amount that is less than 3 mol % relative to the amount of said compound of Formula I.

According to further embodiments, the protic solvent comprises an organic alcohol such as methanol, ethanol, iso-propyl alcohol, or any combination thereof. In some embodiments, the organic alcohol is methanol.

In yet further embodiments, the processes of the invention further can comprise the step of isolating the compound of Formula III by filtration of the reaction mixture.

The reacting of compounds of Formulas I and II can be carried out at any suitable temperature that does not significantly decompose or adversely affect the desired chemical transformation. In some embodiments, the reacting is carried out at an elevated temperature such as from about 30 to about 120, 40 to about 100, 50 to about 75, or about 60° C. Amounts of compounds of Formulas I and II can be provided so as to maximize yield. In some embodiments, the molar ratio of a compound of Formula I to a compound of Formula II is about 1:1. Reaction duration can be sufficiently long to substantially assure reaction completion. In some embodiments, reaction duration can be about 15 minutes to about 2 hours, 15 minutes to about 1 hour, or about 30 minutes.

The reacting according to the present invention optionally can be carried out in the presence of a catalyst such as a cyclic or non-cyclic secondary amine. Some example secondary amines include dialklyamines, diaryl amines, diarylalkyl amines, piperidine, homopiperidine, piperazine, and pyrrolidine. The catalyst can be added in an amount sufficient to accelerate the reaction rate relative to the reaction rate for the process in the absence of catalyst. A suitable amount of catalyst can be about 0.1 to about 50 mol %, about 1 to about 10 mol %, about 5 mol %, about 4 mol %, or about 3 mol %.

The compound of Formula III can be isolated from the reaction mixture by directly filtering the reaction mixture and collecting the filtered solid material. Prior to filtration, the reaction mixture can be cooled to a temperature below room temperature such as from about 0 to about 10, 0 to about 5, or about 0° C.

The term “alkyl”, employed alone or in combination with other terms, is defined herein as, unless otherwise stated, either a straight-chain or branched saturated hydrocarbon moiety. In some embodiments, the alkyl moiety contains 1 to 12, 1 to 10, 1 to 8, 1 to 6, or 1 to 4 carbon atoms. Examples of saturated hydrocarbon alkyl moieties include, but are not limited to, chemical groups such as methyl, ethyl, n-propyl, isopropyl, n-butyl, tert-butyl, isobutyl, sec-butyl; higher homologs such as n-pentyl, n-hexyl, n-heptyl, n-octyl, and the like.

The term “cycloalkyl”, employed alone or in combination with other terms, is defined herein as, unless otherwise stated, a monocyclic, bicyclic, tricyclic, fused, bridged, or spiro monovalent saturated hydrocarbon moiety of 3-10 carbon atoms (e.g., 3-7 carbon atoms). Any suitable ring position of the cycloalkyl moiety can be linked covalently to the defined chemical structure. Examples of cycloalkyl moieties include, but are not limited to, chemical groups such as cyclopropyl, cyclobutyl, cyclopentyl, cyclohexyl, cycloheptyl, norbornyl, adamantyl, spiro[4.5]decanyl, and homologs, isomers, and the like.

The terms “halo” or “halogen”, employed alone or in combination with other terms, is defined herein as, unless otherwise stated, fluoro, chloro, bromo, or iodo.

The term “aryl”, employed alone or in combination with other terms, is defined herein as, unless otherwise stated, an aromatic carbocyclic moiety of up to 14 carbon atoms (e.g., 6-14 carbon atoms), which can be a single ring (monocyclic) or multiple rings (bicyclic, up to three rings) fused together or linked covalently. Any suitable ring position of the aryl moiety can be linked covalently to the defined chemical structure. Examples of aryl moieties include, but are not limited to, chemical groups such as phenyl, 1-naphthyl, 2-naphthyl, dihydronaphthyl, tetrahydronaphthyl, biphenyl, anthryl, phenanthryl, fluorenyl, indanyl, biphenylenyl, acenaphthenyl, acenaphthylenyl, and the like.

The term “arylalkyl”, employed alone or in combination with other terms, is defined herein as, unless otherwise stated, an aryl, as herein before defined, suitably substituted on any open ring position with an alkyl moiety wherein the alkyl chain is a saturated hydrocarbon moiety. In some embodiments, the alkyl moiety has from 1 to 8, 1 to 6, or 1 to 4 carbon atoms. Examples of arylalkyl moieties include, but are not limited to, chemical groups such as benzyl, 1-phenylethyl, 2-phenylethyl, diphenylmethyl, 3-phenylpropyl, 2-phenylpropyl, fluorenylmethyl, and homologs, isomers, and the like.

The term “Het”, employed alone or in combination with other terms, is defined herein as, unless otherwise stated, an heterocyclic ring system having 4-14 ring atoms, which can be a single ring (monocyclic) or multiple rings (bicyclic, up to three rings) fused together or linked covalently. The rings contain from one to four hetero atoms selected from nitrogen (N), oxygen (O), and sulfur (S), wherein the nitrogen or sulfur atom(s) are optionally oxidized, or the nitrogen atom(s) are optionally quaternized. Any suitable ring position of the heterocyclic moiety can be linked covalently to the defined chemical structure. Het can be saturated or unsaturated. Het also can be aromatic or non-aromatic. Examples Het moieties include, but are not limited to, furan, thiophene, pyrrole, N-methylpyrrole, pyrazole, N-methylpyrazole, imidazole, N-methylimidazole, oxazole, isoxazole, thiazole, isothiazole, 1H-tetrazole, 1-methyltetrazole, 1,3,4-oxadiazole, 1H-1,2,4-triazole, 1-methyl-1,2,4-triazole 1,3,4-triazole, 1-methyl-1,3,4-triazole, pyridine, pyrimidine, pyrazine, pyridazine, benzoxazole, benzisoxazole, benzothiazole, benzofuran, benzothiophene, thianthrene, dibenzo[b,d]furan, dibenzo[b,d]thiophene, benzimidazole, N-methylbenzimidazole, indole, indazole, quinoline, isoquinoline, quinazoline, quinoxaline, purine, pteridine, 9H-carbazole, β-carboline, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothienyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, tetrahydrothienyl, tetrahydroquinolinyl, tetrahydroisoquinolinyl, and the like.

The term “Het-alkyl”, employed alone or in combination with other terms, is defined herein as, unless otherwise stated, alkyl substituted by at least one Het. Examples of Het-alkyl moieties include, but are not limited to, chemical groups such as furanylmethyl, thienylethyl, indolylmethyl, and the like.

The term “hydroxyalkyl”, employed alone or in combination with other terms, is defined herein as, unless otherwise stated, a (C₁-C₆) straight chain hydrocarbon, terminally substituted with a hydroxyl group. Examples of hydroxyalkyl moieties include chemical groups such as —CH₂OH, —CH₂CH₂OH, —CH₂CH₂CH₂OH, and higher homologs. Similarly, dihydroxyalkyl indicates an alkyl moiety that is substituted by two hydroxyl groups.

The term “alkoxy”, employed alone or in combination with other terms, is defined herein as, unless otherwise stated, —O-alkyl. Examples of alkoxy moieties include, but are not limited to, chemical groups such as methoxy, ethoxy, isopropoxy, sec-butoxy, tert-butoxy, and homologs, isomers, and the like.

The term “alkylthio” or “thioalkoxy” employed alone or in combination with other terms, is defined herein as, unless otherwise stated, —S-alkyl. Examples of alkylthio moieties include, but are not limited to, chemical groups such as methylthio, ethylthio, isopropylthio, sec-butylthio, tert-butylthio, and homologs, isomers, and the like.

The compounds of the present invention can contain an asymmetric atom, and some of the compounds can contain one or more asymmetric atoms or centers, which, thus, can give rise to optical isomers (enantiomers) and diastereomers. The present invention includes such optical isomers (enantiomers) and diastereomers (geometric isomers), as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, and include, but are not limited to, diastereomeric salt formation, kinetic resolution, and asymmetric synthesis. It is also understood that this invention encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

As used herein, the term “reacting” refers to the bringing together of designated chemical reactants such that a chemical transformation takes place generating a compound different from any initially introduced into the system. Reacting can take place in the presence or absence of solvent.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatograpy (HPLC) or thin layer chromatography.

The reactions of the processes described herein can be carried out in suitable solvents, which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected. In some embodiments, reactions can be carried out in the absence of solvent, such as when at least one of the reagents is a liquid or gas.

The term protic solvent refers to a solvent that is capable of functioning as an acid for purposes of protonating any unreacted, strongly basic reaction intermediates. Suitable protic solvents can include, by way of example and without limitation, water, methanol, ethanol, 2-nitroethanol, 2-fluoroethanol, 2,2,2-trifluoroethanol, ethylene glycol, 1-propanol, 2-propanol, 2-methoxyethanol, 1-butanol, 2-butanol, 1-butyl alcohol, t-butyl alcohol, 2-ethoxyethanol, diethylene glycol, 1-, 2-, or 3-pentanol, neopentyl alcohol, t-pentyl alcohol, diethylene glycol monomethyl ether, diethylene glycol monoethyl ether, cyclohexanol, benzyl alcohol, phenol, or glycerol.

The term non-protic solvent refers to an organic solvent that is not readily deprotonated in the presence of a strongly basic reactant. Suitable non-protic solvents can include, by way of example and without limitation, ethers, dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethyl sulfoxide, propionitrile, ethyl formate, methyl acetate, hexachloroacetone, acetone, ethyl methyl ketone, ethyl acetate, sulfolane, N,N-dimethylpropionamide, tetramethylurea, nitromethane, nitrobenzene, or hexamethylphosphoramide.

The term non-protic solvent also refers to many ether solvents including: diethoxymethane, tetrahydrofuran, 1,3-dioxane, 1,4-dioxane, furan, diethyl ether, tetrahydropyran, diisopropyl ether, dibutyl ether, ethylene glycol dimethyl ether, ethylene glycol diethyl ether, diethylene glycol dimethyl ether, diethylene glycol diethyl ether, triethylene glycol dimethyl ether, anisole, and t-butyl methyl ether.

The reactions of the processes described herein can be carried out at appropriate temperatures, which can be readily determined by the skilled artisan. Reaction temperatures will depend on, for example, the melting and boiling points of the reagents and solvent, if present; the thermodynamics of the reaction (e.g., vigorously exothermic reactions typically are carried out at reduced temperatures); and the kinetics of the reaction (e.g., a high activation energy barrier typically necessitates elevated temperatures). “Elevated temperatures” refers to temperatures above room temperature (about 20 ° C.) and “reduced temperatures” refers to temperatures below room temperature.

The reactions of the processes described herein can be carried out in air or under an inert atmosphere. Typically, reactions containing reagents or products that are substantially reactive with air can be carried out using air-sensitive synthetic techniques that are well known to the skilled artisan.

It is appreciated that certain features of the invention, which are, for clarity, described in the context of separate embodiments, also can be provided in combination in a single embodiment. Conversely, various features of the invention which are, for brevity, described in the context of a single embodiment, also can be provided separately or in any suitable subcombination.

The invention will be described in greater detail by way of specific examples. The following examples are offered for illustrative purposes, and are not intended to limit the invention in any manner. Those of skill in the art will readily recognize a variety of noncritical parameters, which can be changed or modified to yield essentially the same results.

### EXAMPLE 1

### Preparation of 2-cyano-3-naphthalen-1-yl-acrylic Acid Methyl Ester (Compound 3)

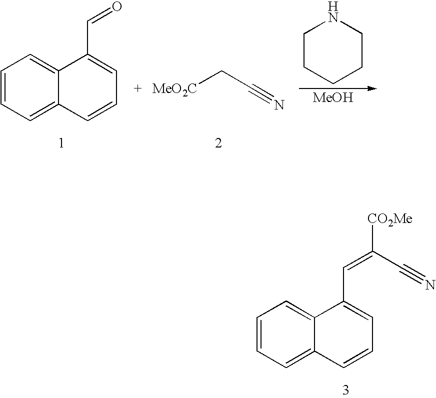

To a 500 ml flask equipped with a heating mantle, a mechanical stirrer, a thermometer and a condenser were added 162 g methanol, 45 g 1-naphthaldehyde (0.29 mol), 28.5 g methyl cyanoacetate (0.29 mol) and 0.5 g piperidine (0.006 mol). The mixture was heated to 60° C. for 30 min and then cooled to 1° C. The solid was filtered, then washed with 100 g of methanol and dried in an oven under vacuum. Yield: 63.3 g (92.5%).

In a variation on the above procedure, the above reaction was carried out in the absence of piperidine for 24 hours at room temperature, and in another variation on the above procedure, the above reaction was carried out for 10 hours at 50-60° C., both with similar results.

Various modifications of the invention, in addition to those described herein, will be apparent to those skilled in the art from the foregoing description. Such modifications also are intended to fall within the scope of the appended claims. Each reference cited in the present application, including but not limited to printed publications, patents and patent applications, is incorporated herein by reference in its entirety.

1. A process for preparing a compound of Formula III: 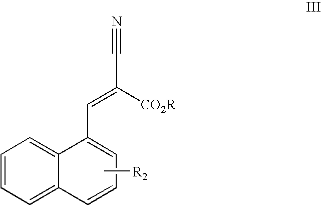
wherein:
R is C₁-₆ alkyl or benzyl;
R₂ is hydrogen, halogen, C₁₋₆alkyl, C₁₋₆ alkoxy, nitro, cyano, aryl, CF₃, OCF₃, NR₄R₅ or OH;
R₄ and R₅ are each, independently, hydrogen, C₁₋₆ alkyl, aryl, arylalkyl having 1-6 carbon atoms in the alkyl moiety, Het-alkyl having 1-6 carbon atoms in the alkyl moiety, hydroxyalkyl of 1-6 carbons, dihydroxyalkyl of 1-6 carbons, or cycloalkyl of 3-7 carbons;
or R₄ and R₅ together with the N atom to which they are attached form a 5- or 6-membered heterocycle; and
Het is a heterocyclic ring system of 4-14 ring atoms comprising one to four ring-forming heteroatoms;
comprising reacting a compound of Formula I: 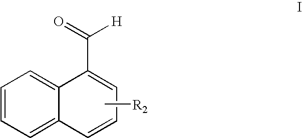
in a protic solvent with a compound of Formula II:

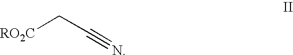

2. The process of claim 1 wherein R is C₁₋₆ alkyl and R₂ is hydrogen or C₁₋₄ alkyl.

3. The process of claim 1 wherein R is C₁₋₄ alkyl and R₂ is hydrogen.

4. The process of claim 1 wherein R is methyl and R₂ is hydrogen.

5. The process of claim 1 wherein said reacting is carried out in the presence of a catalytic amount of secondary amine.

6. The process of claim 5 wherein said secondary amine is pyrrolidine, piperidine, or homopiperidine.

7. The process of claim 5 wherein said secondary amine is present in less than 10 mol % relative to the amount of said compound of Formula I.

8. The process of claim 5 wherein said secondary amine is present in less than 3 mol % relative to the amount of said compound of Formula I.

9. The process of claim 1 wherein said protic solvent comprises an organic alcohol.

10. The process of claim 9 wherein said organic alcohol is methanol, ethanol, iso-propyl alcohol, or combination thereof.

11. The process of claim 9 wherein said organic alcohol is methanol.

12. The process of claim 1 further comprising the step of isolating said compound of Formula III by filtration of the reaction mixture resulting from said reaction.
